(19) United States
(12) Patent Application Publication
(10) Pub. No.: US 2026/0151590 A1
(43) Pub. Date: Jun. 4, 2026

(54) CATHETER

(71) Applicant: KANEKA CORPORATION, Osaka (JP)

(72) Inventor: Tomoya Kato, Osaka (JP)

(73) Assignee: KANEKA CORPORATION, Osaka (JP)

(21) Appl. No.: 19/459,250

(22) Filed: Jan. 26, 2026

Related U.S. Application Data
(63) Continuation of application No. PCT/JP2024/024768, filed on Jul. 9, 2024.

(30) Foreign Application Priority Data
Aug. 1, 2023 (JP) 2023-125555

(51) Int. Cl. A61M 25/00; A61M 25/01

(52) U.S. Cl. CPC A61M 25/0026; A61M 25/0043; A61M 25/0108; A61M 2025/0037; A61M 2025/0059; A61M 2205/32

(57) ABSTRACT

A catheter includes a first tubular member; a second tubular member adjacent to the first tubular member; and an outer member including a portion that encloses at least a portion of the first tubular member and the second tubular member. The distal end of the first tubular member is located on the proximal side of the distal end of the second tubular member, the proximal end of the first tubular member is located on the proximal side of the proximal end of the second tubular member, and the distal end of the outer member is located on the distal side of the distal end of the second tubular member. The outer member includes a first thick portion, a first thin portion located on the proximal side of the first thick portion, and a second thick portion located on the proximal side of the first thin portion.

### TECHNICAL FIELD

One or more embodiments of the present invention relate to a catheter.

### BACKGROUND

A catheter that can be inserted into a blood vessel or the like is used to examine and/or treat a lesion site of a human body. As such a catheter, for example, PTL 1 discloses a catheter including: a catheter body that includes a hollow coil body including a plurality of wound or twisted metal wires, at least one of which has a larger diameter than the other wires, an inner resin layer covering the inner circumferential surface of the coil body, and an outer resin layer covering, in a substantially circular shape, the outer circumferential surface of the coil body such that the outer surface of the larger-diameter wire of the coil body is not exposed to the outside; a tip including a cylindrical body made of a resin, the tip having an inner hole in communication with a hollow portion of the catheter body and being provided at the distal end of the catheter body; an inner-layer extension portion in which the inner resin layer extends integrally from the distal end of the catheter body toward the tip and forms an inner circumferential portion of a proximal end portion of the tip; and a braid body including a plurality of braided wires, the braid body being disposed, in a state of being positioned to extend in an axial direction across the boundary between a proximal end portion of the tip and a distal end portion of the catheter body, at each of the inner-layer extension portion in the tip and the inner resin layer at at least a portion of the catheter body near the distal end.

### PATENT LITERATURE

PTL 1: Japanese Unexamined Patent Application Publication No. 2011-125737

### SUMMARY

As a result of studies by the present inventor(s), it has been found that, when an existing catheter is to be inserted into a curved portion of a blood vessel or the like, it is sometimes difficult to insert the catheter. Specifically, it has been found that, when the catheter is to be inserted using a guidewire to a target site deeper than a curved portion in the body, the catheter is excessively curved, and the inner surface of the catheter and the guidewire are likely to come into contact with each other. As a result, it is difficult to insert the catheter. One or more embodiments of the present invention have been made in view of the above and a catheter that can be easily inserted into a curved portion in the body is provided.

A catheter according to one or more embodiments of the present invention capable of addressing the above is as follows.
[1] A catheter including:
a first tubular member extending in a longitudinal direction;
a second tubular member extending in the longitudinal direction and adjacent to the first tubular member in a radial direction; and
an outer member extending in the longitudinal direction and including a portion that encloses at least a portion of the first tubular member and at least a portion of the second tubular member,
wherein a distal end of the first tubular member is located on a proximal side of a distal end of the second tubular member,
wherein a proximal end of the first tubular member is located on a proximal side of a proximal end of the second tubular member,
wherein a distal end of the outer member is located on a distal side of the distal end of the second tubular member, and
wherein the outer member includes:
a first thick portion,
a first thin portion located on a proximal side of the first thick portion and thinner than the first thick portion, and
a second thick portion located on a proximal side of the first thin portion and thicker than the first thin portion.

As a result of studies by the present inventor(s), it has been found that a catheter that has, at a distal end portion of the catheter, an outer layer that becomes thinner toward the distal end thereof may be excessively curved when passing through a curved portion in the body. On the other hand, in the catheter according to the embodiment, since the outer member includes the first thick portion, the first thin portion, and the second thick portion in order from the distal side, it is possible to prevent the distal end portion of the catheter from being excessively curved. As a result, when the catheter is inserted using a guidewire into a curved portion in the body, contact between the inner surface of the catheter and the guidewire can be reduced, and thus, the catheter can be easily inserted into the curved portion.

Further, the catheter according to an embodiment may be the catheter in any one of [2] to [15] below.
[2] The catheter according to [1], wherein the first thick portion includes the distal end of the outer member.
[3] The catheter according to [1] or [2], wherein the first thick portion and the first thin portion are adjacent to each other, and the first thin portion and the second thick portion are adjacent to each other.
[4] The catheter according to [1] to [3], wherein the second thick portion includes a portion thicker than the first thick portion.
[5] The catheter according to [1] to [4], wherein the second thick portion includes a raised portion raised in a direction away from the second tubular member.
[6] The catheter according to [1] to [5], wherein a length of the first thin portion in the longitudinal direction is longer than a length of the first thick portion in the longitudinal direction.
[7] The catheter according to [1] to [6], wherein a length of the first thin portion in the longitudinal direction is longer than a length of the second thick portion in the longitudinal direction.
[8] The catheter according to [1] to [7], wherein the second tubular member includes a radiopaque marker located on a distal side of a proximal end of the first thin portion and on a proximal side of a distal end of the first thin portion.
[9] The catheter according to [1] to [8], wherein the first thick portion includes a portion that becomes thinner toward the distal end of the outer member.
[10] The catheter according to [1] to [9],
wherein the first thin portion includes
a first portion,
a second portion located on a proximal side of the first portion and thinner than the first portion, and
a third portion located on a proximal side of the second portion and thicker than the second portion.
[11] The catheter according to any one of [1] to [10],
wherein the outer member includes a columnar body extending in the longitudinal direction and containing a resin, and
wherein at least a portion of the first tubular member and at least a portion of the second tubular member are disposed in the columnar body.
[12] The catheter according to any one of [1] to [10], wherein the outer member has a tubular shape and has a lumen extending in the longitudinal direction, and at least a portion of the first tubular member and at least a portion of the second tubular member are disposed in the lumen.
[13] The catheter according to [1] to [12], wherein the outer member includes a body portion, a first distal-end portion, and a second distal-end portion, wherein: the body portion encloses a portion of the first tubular member and a portion of the second tubular member, the first distal-end portion is located on a distal side of the body portion and encloses a distal end portion of the first tubular member, the second distal-end portion is located on a distal side of the body portion and encloses a distal end portion of the second tubular member, and the second distal-end portion contains a material different from a material contained in the first distal-end portion and from a material contained in the body portion.
[14] The catheter according to any one of [1] to [13], wherein the first tubular member and the second tubular member are fixed to each other in the radial direction.
[15] The catheter according to any one of [1] to [14], wherein a handle portion is disposed at a proximal end portion of the first tubular member.

According to one or more embodiments of the present invention, with the above-described configuration, it is possible to provide a catheter that is easily inserted into a curved portion in the body.

### BRIEF DESCRIPTION OF DRAWINGS

FIG. 1 is a side view of a catheter according to a first embodiment.

FIG. 2 is a longitudinal sectional view of the region R1 in FIG. 1.

FIG. 3 is a longitudinal sectional view of the region R2 in FIG. 1.

FIG. 4 is a schematic view illustrating an example of the catheter in FIG. 1 when being inserted into a curved portion in the body.

FIG. 5 is a cross-sectional view of the catheter taken along line A-A in FIG. 1.

FIG. 6 is a longitudinal sectional view of a modification of the region R1 in FIG. 1.

FIG. 7 is a side view of a catheter according to a second embodiment.

FIG. 8 is a longitudinal sectional view of the region R1 in FIG. 7.

FIG. 9 is a longitudinal sectional view of the region R2 in FIG. 7.

FIG. 10 is a cross-sectional view of the catheter taken along line A-A in FIG. 7.

### DETAILED DESCRIPTION

Hereinafter, one or more embodiments of the present invention will be described more specifically on the basis of the following embodiments. However, needless to say, the present invention is not limited by the following embodiments and can be naturally carried out with appropriate changes within the scope that can be adapted to the gist described above and below, and all of them are included in the technical scope of the present invention. In each of the drawings, reference signs and the like of members may be omitted for convenience. In such a case, the specification and other drawings are referred to. In addition, the dimensions of various members in the drawings may be different from actual dimensions thereof for facilitating understanding of the features of one or more embodiments of the present invention.

A catheter according to an embodiment is a catheter including a first tubular member extending in a longitudinal direction; a second tubular member extending in the longitudinal direction and adjacent to the first tubular member in a radial direction; and an outer member extending in the longitudinal direction and including a portion that encloses at least a portion of the first tubular member and at least a portion of the second tubular member. The distal end of the first tubular member is located on the proximal side of the distal end of the second tubular member, the proximal end of the first tubular member is located on the proximal side of the proximal end of the second tubular member, the distal end of the outer member is located on the distal side of the distal end of the second tubular member, and the outer member includes a first thick portion, a first thin portion located on the proximal side of the first thick portion and thinner than the first thick portion, and a second thick portion located on the proximal side of the first thin portion and thicker than the first thin portion.

As a result of studies by the present inventor(s), it has been found that a catheter that has, at a distal end portion of the catheter, an outer layer that becomes thinner toward the distal end thereof may be excessively curved when passing through a curved portion in the body. On the other hand, in the catheter according to the embodiment, since the outer member includes the first thick portion, the first thin portion, and the second thick portion in order from the distal side, it is possible to prevent the distal end portion of the catheter from being excessively curved. As a result, when the catheter is inserted using a guidewire into a curved portion in the body, contact between the inner surface of the catheter and the guidewire can be reduced, and thus, the catheter can be easily inserted into the curved portion.

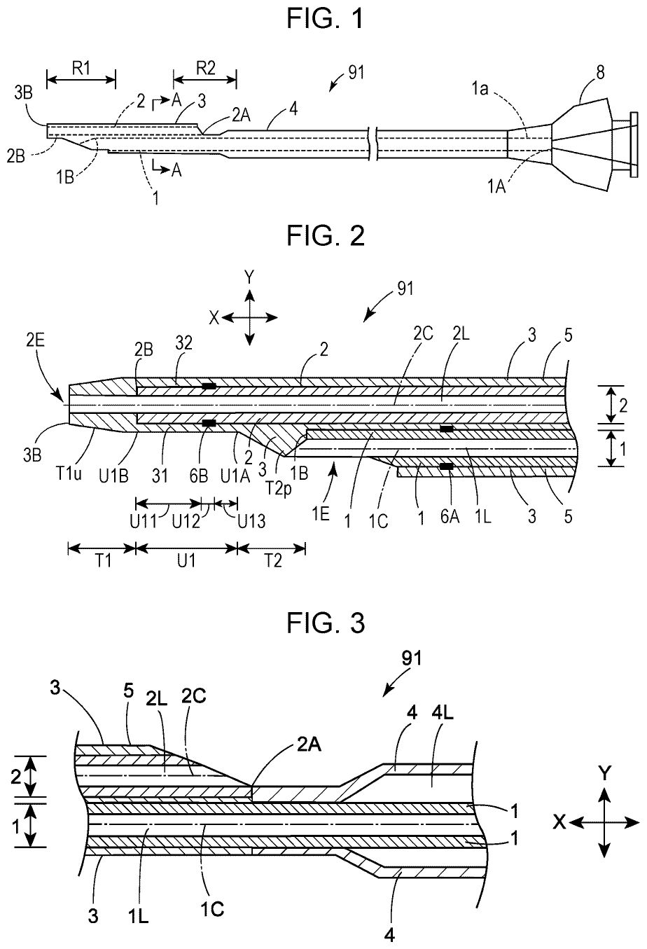

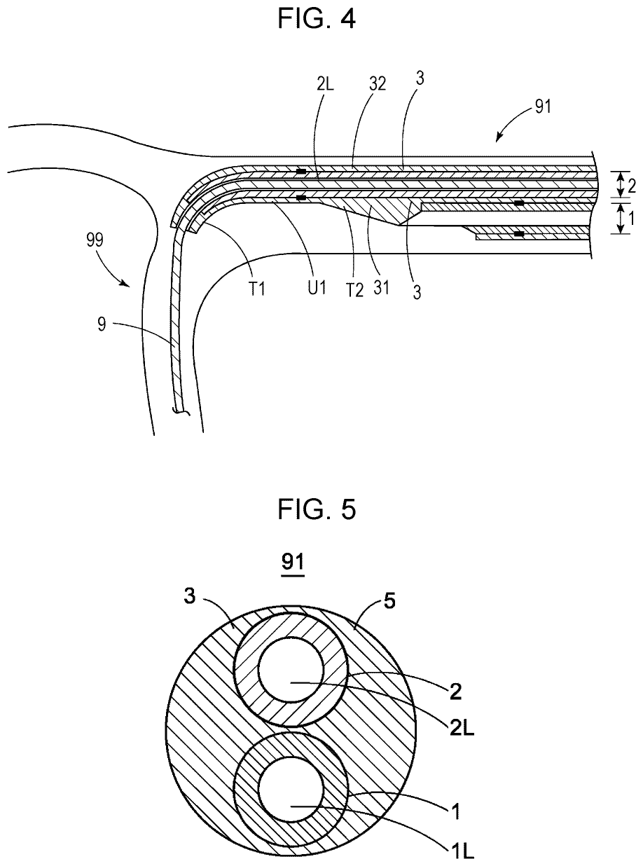

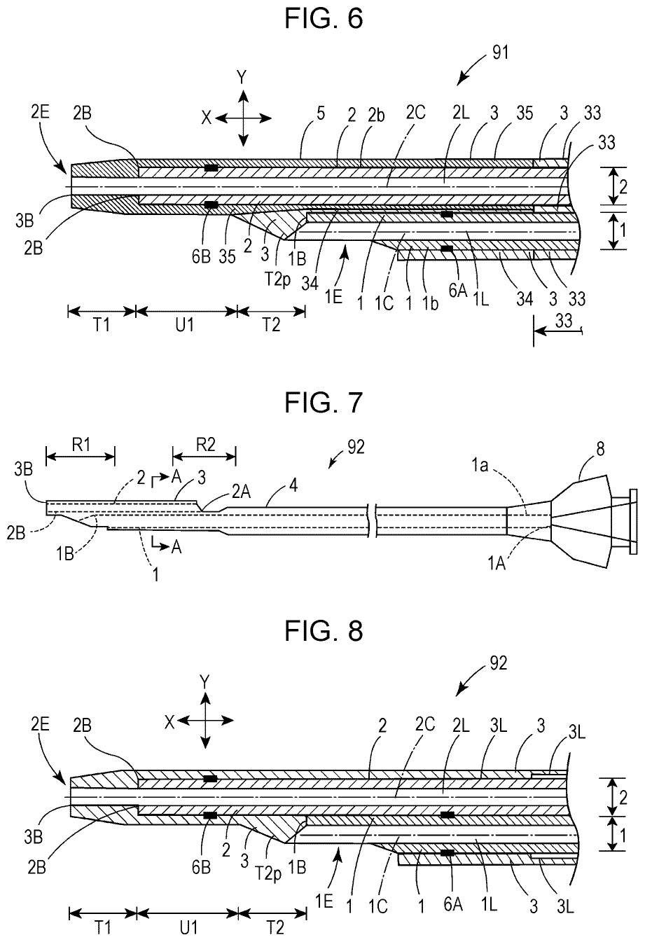

Hereinafter, a catheter according to a first embodiment will be described with reference to FIGS. 1 to 6. FIG. 1 is a side view of a catheter according to the first embodiment. FIG. 2 is a longitudinal sectional view of the region R1 in FIG. 1. FIG. 3 is a longitudinal sectional view of the region R2 in FIG. 1. FIG. 4 is a schematic view illustrating an example of the catheter in FIG. 1 when being inserted into a curved portion in the body. FIG. 5 is a cross-sectional view of the catheter taken along line A-A in FIG. 1. FIG. 6 is a longitudinal sectional view of a modification of the region R1 in FIG. 1.

As illustrated in FIGS. 1, 2, and 3, a catheter 91 according to the first embodiment includes a first tubular member 1 extending in a longitudinal direction X, and a second tubular member 2 extending in the longitudinal direction X and adjacent to the first tubular member 1 in a radial direction Y. Specifically, it is preferable that an outer surface of the first tubular member 1 and an outer surface of the second tubular member 2 are adjacent to each other in the radial direction Y. The first tubular member 1 may have a lumen 1L extending in the longitudinal direction X. The second tubular member 2 may have a lumen 2L extending in the longitudinal direction X. For example, by inserting a guidewire into each of the lumen 1L and the lumen 2L, the catheter 91 can be guided in multiple directions. Therefore, the catheter 91 can be easily inserted into various branch portions of, for example, coronary arteries. In addition, by inserting a search wire into the lumen 1L or the lumen 2L, it is possible to easily search for an entrance or the like of a stenotic portion, which is a lesioned portion. It is preferable to insert the search wire into the lumen 1L. A distal end portion of the search wire may be bent.

Each of the first tubular member 1 and the second tubular member 2 may have one or a plurality of layers extending in the longitudinal direction X or may have a plurality of layers. The plurality of layers may include an inner layer and an outer layer that encloses at least a portion of the inner layer. The plurality of layers may further include an intermediate layer positioned between the inner layer and the outer layer. The number of intermediate layers is not limited to one and may be two or more. Each of the plurality of layers may contain a material different from a material of a layer adjacent thereto. The inner layer of the first tubular member 1 and the inner layer of the second tubular member 2 each may contain at least one resin selected from the group consisting of a polyolefin-based resin and a fluorine-based resin. Consequently, the slidability of a guidewire inserted into the lumen 1L and the lumen 2L can be improved. The polyolefin-based resin may be polyethylene, polypropylene, an ethylene-propylene copolymer, or a mixture thereof. The polyethylene may be high-density polyethylene having a specific gravity of 0.942 or greater. The fluorine-based resin may be polytetrafluoroethylene, a tetrafluoroethylene-perfluoroalkyl vinyl ether copolymer, a tetrafluoroethylene-hexafluoropropylene copolymer, a tetrafluoroethylene-ethylene copolymer, polyvinylidene fluoride, polychlorotrifluoroethylene, an ethylene-chlorotrifluoroethylene copolymer, or a mixture thereof. The inner layer of the first tubular member 1 and the inner layer of the second tubular member 2 may each contain at least one selected from the group consisting of a polyamide-based resin, a polyester-based resin, a polyurethane-based resin, a polyolefin-based resin, a fluorine-based resin, a vinyl chloride resin, a silicone-based resin, and natural rubber. The inner layer of the first tubular member 1 and the inner layer of the second tubular member 2 may contain resins different from each other, preferably contain the same resin and more preferably are made of the same resin.

Each of the outer layer of the first tubular member 1 and the outer layer of the second tubular member 2 may contain a flexible resin. The flexible resin may contain at least one selected from the group consisting of polyethylene, polyurethane, a polyurethane-based thermoplastic elastomer, a styrene-based thermoplastic elastomer, a polyamide elastomer, and polyamide or contain at least one selected from the group consisting of polyurethane and a polyamide elastomer. The polyethylene may be low-density polyethylene having a specific gravity of 0.91 to 0.92.

The Shore D hardness of the material of each outer layer may be less than or equal to, or may be less than, the Shore D hardness of the material of each inner layer. Consequently, the flexibility of the catheter 91 can be improved. Shore D hardness may be measured, for example, with a Durometer Type D in accordance with ISO7619.

Each intermediate layer may contain a material different from the material of each inner layer or each outer layer, or may contain the same material as the material of each inner layer or each outer layer.

Each of the inner layer, the intermediate layer, and the outer layer may be fixed to a layer adjacent thereto. Consequently, each layer is less likely to be wrinkled or twisted. Examples of the fixing method include welding and adhesion.

The first tubular member 1 may include a reinforcing member. Consequently, it is possible to easily prevent puncture of the first tubular member 1 by a wire. The second tubular member 2 may not be provided with a reinforcing member. Consequently, the flexibility of the second tubular member 2 can be easily improved.

The reinforcing member may be a braided body in which a wire material is braided, a coil body in which a wire material is spirally wound, or a combination thereof. Consequently, it is possible to easily prevent puncture of the first tubular member 1 by a guidewire. In particular, the braided body easily improves the rigidity of the reinforcing member. Thus, the reinforcing member may be the braided body. Examples of the wire material included in the reinforcing member include a metal wire and fiber. The metal wire may contain stainless steel, titanium, a nickel-titanium alloy, a cobalt-chromium alloy, a tungsten alloy, or alloys thereof. In particular, stainless steel is more preferable. The metal wire may be a single wire or a stranded wire. As the fiber, polyarylate fiber, aramid fiber, ultra-high-molecular-weight polyethylene fiber, PBO fiber, carbon fiber, or a mixed fiber thereof is preferable. The fiber may be monofilaments or multifilaments. The reinforcing member may contain a radiopaque substance so that the position of the first tubular member 1 can be easily confirmed under X-ray fluoroscopy or the like. The radiopaque substance may be at least one selected from the group consisting of lead, barium, iodine, tungsten, gold, platinum, iridium, a platinum-iridium alloy, stainless steel, titanium, a cobalt-chromium alloy, palladium, bismuth, and tantalum.

Although not illustrated, the distal end of the reinforcing member may be located on the proximal side of the proximal end of a first opening portion 1E and on the distal side of a proximal end 2A of the second tubular member 2. On the other hand, the proximal end of the reinforcing member may be located on the proximal side of the proximal end 2A of the second tubular member 2 or may be located in a handle portion 8. Consequently, it is possible to easily prevent puncture of the first tubular member 1 by a guidewire.

As illustrated in FIG. 5, the outer surface of the first tubular member 1 and the outer surface of the second tubular member 2 may be not in contact with each other or may be in contact with and fixed to each other. Examples of the fixing method include welding and adhesion. When the outer surface of the first tubular member 1 and the outer surface of the second tubular member 2 are not in contact with each other, the outer surfaces may be fixed to each other via the resin of an outer member 3. As illustrated in FIG. 5, in a cross-section of the catheter 91 in the radial direction Y, the shape of each of the lumen 1L and the lumen 2L may be a circular shape, an elliptical shape, an arcuate shape, or a combination thereof, or may be a circular shape or an elliptical shape. Consequently, the catheter 91 is easily curved. In the catheter 91, it is preferable that the lumen 1L and the lumen 2L are not in communication with each other. In a cross-section in the radial direction Y, the area of the lumen 1L may be larger than the area of the lumen 2L, may be smaller than the area of the lumen 2L, or may be the same as the area of the lumen 2L.

As illustrated in FIG. 2, a distal end 1B of the first tubular member 1 is located on the proximal side of a distal end 2B of the second tubular member 2. Consequently, the flexibility of a portion of the catheter 91 on the distal side of the distal end 1B of the first tubular member 1 can be improved.

As illustrated in FIGS. 1 and 3, a proximal end 1A of the first tubular member 1 is located on the proximal side of the proximal end 2A of the second tubular member 2. Consequently, a portion of the catheter 91 on the proximal side of the proximal end 2A of the second tubular member 2 can be formed to have, for example, a double-tube structure (coaxial structure) so as to have a structure different from the structure, in which the first tubular member 1 and the second tubular member 2 overlap each other in the radial direction Y, on the distal side of the proximal end 2A of the second tubular member 2 and so as to be exert a different function.

As illustrated in FIGS. 2, 3, and 5, the catheter 91 includes the outer member 3 extending in the longitudinal direction X and including a portion that encloses at least a portion of the first tubular member 1 and at least a portion of the second tubular member 2. The outer member 3 can protect the first tubular member 1 and the second tubular member 2 and can easily prevent puncture by a guidewire from inside the catheter 91.

The outer member 3 may contain at least one selected from the group consisting of polyethylene, polyurethane, a polyurethane-based thermoplastic elastomer, a styrene-based thermoplastic elastomer, a polyamide elastomer, and polyamide. In particular, at least one selected from the group consisting of polyurethane and a polyamide elastomer is preferable.

As illustrated in FIG. 5, the outer member 3 may include a columnar body 5 extending in the longitudinal direction X and containing a resin, and at least a portion of the first tubular member 1 and at least a portion of the second tubular member 2 may be disposed in the columnar body 5. That is, it is preferable that at least a portion of each of the first tubular member 1 and the second tubular member 2 is sealed by the columnar body 5 extending in the longitudinal direction X and containing a resin. Consequently, even when the catheter 91 is curved, the shapes of the lumen 1L and the lumen 2L extending in the longitudinal direction X are easily maintained. The columnar body 5 may be disposed at least between the proximal end 2A and the distal end 2B of the second tubular member 2. The resin of the columnar body 5 may present between the first tubular member 1 and the second tubular member 2.

The shape of the columnar body 5 may be a columnar shape, an elliptic columnar shape, or a polygonal columnar shape with rounded corners or may be a columnar shape or an elliptic columnar shape. Consequently, the catheter 91 is easily inserted into the body.

The columnar body 5 may be formed by covering the first tubular member 1 and the second tubular member 2, which are overlapped in the radial direction Y, with a resin tube and heating and welding the resin tube or the columnar body 5 may be formed by performing injection-molding with respect to the first tubular member 1 and the second tubular member 2 overlapped in the radial direction Y.

As illustrated in FIG. 2, a distal end 3B of the outer member 3 is located on the distal side of the distal end 2B of the second tubular member 2. Such an arrangement makes it easy to form a first thick portion T1 of the outer member 3.

At least a portion of the inner surface of the outer member 3 may be fixed to outer surfaces of the first tubular member 1 and the second tubular member 2. Consequently, the outer member 3 is unlikely to be wrinkled or twisted. Examples of the fixing method include welding and adhesion.

The outer member 3 includes the first thick portion T1, a first thin portion U1 is located on the proximal side of the first thick portion T1 and thinner than the first thick portion T1, and a second thick portion T2 located on the proximal side of the first thin portion U1 and thicker than the first thin portion U1. Consequently, excessive curving of the distal end portion of the catheter 91 can be prevented. For example, excessive curving when a guidewire 9 is inserted as illustrated in FIG. 4 into the lumen 2L of the second tubular member 2 and the catheter 91 is inserted into a curved portion 99 in the body along the guidewire 9 is prevented, and as a result, the contact between the inner surface of the catheter 91 and the guidewire 9 is reduced. Therefore, the catheter 91 can be easily inserted into the curved portion 99 in the body. The curved portion 99 in the body is, for example, an artery such as a coronary artery.

As illustrated in FIG. 2, it is preferable that the above thickness relationship among the first thick portion T1, the first thin portion U1, and the second thick portion T2 is satisfied in a cross-section (hereinafter, referred to as “cross-section X”) in the longitudinal direction X including a central axis 1C of the first tubular member 1 and a central axis 2C of the second tubular member 2. Specifically, in the cross-section X, it is preferable that the outer member 3 includes a first region 31 on a side close to the first tubular member 1 in the radial direction Y and a second region 32 on a side far from the first tubular member 1 in a portion adjacent to the second tubular member 2, and the above thickness relationship is satisfied at least in the first region 31. Consequently, excessive curving in the direction from the second region 32 toward the first region 31 can be easily prevented. Further, although not illustrated, it is more preferable that the above thickness relationship is satisfied in each of the first region 31 and the second region 32. Consequently, it is possible to easily prevent excessive curving in the direction from the first region 31 toward the second region 32 and in the direction from the second region 32 toward the first region 31.

The first thick portion T1 may include the distal end 3B of the outer member 3. Consequently, excessive curving of the distal end 3B of the outer member 3 and the vicinity thereof can be easily prevented. Further, it is preferable that the first thick portion T1 is located on the distal side of the distal end 2B of the second tubular member 2. Consequently, the first thick portion T1 is easily curved to some extent.

The first thick portion T1 and the first thin portion U1 may be adjacent to each other, and the first thin portion U1 and the second thick portion T2 may be adjacent to each other. Consequently, the first thick portion T1, the first thin portion U1, and the second thick portion T2 are easily integrally curved, and it is possible to easily prevent excessive curving.

The second thick portion T2 may include a portion thicker than the first thick portion T1. Consequently, the pushability can be improved.

The second thick portion T2 may include a raised portion T2p that is raised in a direction away from the second tubular member 2. Although not illustrated, for example, by bringing a wire inserted from the proximal side of the first tubular member 1 into contact with the raised portion T2p, the distal end of the wire can be guided in a direction different from the direction of the distal end of a wire inserted into the second tubular member 2. Therefore, it is possible to easily perform treatment on lesions in various bifurcated portions. In addition, it is thus possible to make the wires less likely to be entangled. The raised portion T2p may be adjacent to the distal end 1B of the first tubular member 1. Consequently, a wire that has emerged from the first opening portion 1E is easily guided by the raised portion T2p.

The raised portion T2p may include, in the cross-section X, a first inclined portion inclined in a direction away from the central axis 2C of the second tubular member 2 toward the distal side and a second inclined portion inclined so as to approach the central axis 2C of the second tubular member 2 toward the distal side. The second inclined portion may be located on the distal side of the first inclined portion. The second inclined portion allows the catheter 91 to be easily inserted into the body. The first inclined portion makes it easy to change the direction of a wire inserted from the proximal side of the first tubular member 1.

The raised portion T2p may be located in the first region 31 of the outer member 3. Consequently, excessive curving in the direction from the second region 32 toward the first region 31 can be easily prevented.

The length of the first thin portion U1 in the longitudinal direction X may be longer than the length of the first thick portion T1 in the longitudinal direction X. Consequently, the first thin portion U1 is easily curved as a whole, and thus, local bending of the first thin portion U1 is easily avoided. The length of the first thin portion U1 in the longitudinal direction X may be not less than 1.5 times and not greater than 8 times or not less than 2.0 times and not greater than 5 times the length of the first thick portion T1 in the longitudinal direction X. The length of the first thin portion U1 in the longitudinal direction X may be not less than 2 mm and not greater than 8 mm or not less than 3 mm and not greater than 6 mm. It is preferable that the first thin portion U1 does not include an inclined portion that is inclined in a direction away from the central axis 2C of the second tubular member 2 toward the distal end 3B of the outer member 3. Consequently, the first thin portion U1 is more easily curved as a whole.

The length of the first thin portion U1 in the longitudinal direction X may be longer than the length of the second thick portion T2 in the longitudinal direction X. Consequently, the first thin portion U1 is easily curved as a whole, and thus, local bending of the first thin portion U1 is easily avoided. The length of the first thin portion U1 in the longitudinal direction X may be not less than 1.5 times and not greater than 6 times or not less than 2.0 times and not greater than 3 times the length of the second thick portion T2 in the longitudinal direction X.

It is preferable that the second tubular member 2 includes a radiopaque marker 6B located on the distal side of a proximal end U1A of the first thin portion U1 and on the proximal side of the distal end U1B. Consequently, when the catheter 91 is inserted into the curved portion 99 in the body, the position of a curved portion of the catheter 91 can be easily grasped, and thus, the insertion can be easily performed. Consequently, it is also easy to prevent the first thin portion U1 from being excessively curved.

The radiopaque marker may have a ring shape. The radiopaque marker may contain a radiopaque substance or may be made of a radiopaque substance. The radiopaque substance may be at least one selected from the group consisting of lead, barium, iodine, tungsten, gold, platinum, iridium, a platinum-iridium alloy, stainless steel, titanium, a cobalt-chromium alloy, palladium, bismuth, and tantalum.

It is preferable that the first thick portion T1 includes a thinned portion T1u that becomes thinner toward the distal end 3B of the outer member 3. Consequently, the first thick portion T1 is easily inserted into the body. The shape of an outer surface of the portion T1u in the cross-section X may be linear or curved or may be linear. When the outer surface is linear, the portion T1u has a so-called tapered shape.

In FIG. 2, the inner diameter of the outer member 3 on the distal side of the distal end 2B of the second tubular member 2 is the same length as the inner diameter of the second tubular member 2 at the distal end 2B. However, the inner diameter of the outer member 3 on the distal side of the distal end 2B may be longer than the inner diameter of the second tubular member 2 at the distal end 2B. Consequently, the inner surface of the first thick portion T1 is less likely to come into contact with the guidewire 9.

The first thin portion U1 may include a first portion U11, a second portion U12 located on the proximal side of the first portion U11 and thinner than the first portion U11, and a third portion U13 located on the proximal side of the second portion U12 and thicker than the second portion U12. The first portion U11 and the third portion U13, which are relatively thick, can prevent the first thin portion U1 from being excessively curved.

The first portion U11 and the second portion U12 may be adjacent to each other, and the second portion U12 and the third portion U13 may be adjacent to each other. Consequently, the first portion U11, the second portion U12, and the third portion U13 are easily curved integrally, and it is possible to easily prevent excessive curving.

It is preferable that the above-described thickness relationship among the first portion U11, the second portion U12, and the third portion U13 is satisfied in the cross-section X as illustrated in FIG. 2. Specifically, in the cross-section X, it is preferable that the thickness relationship is satisfied at least in the first region 31. Consequently, excessive curving in the direction from the second region 32 toward the first region 31 can be easily prevented. Further, it is more preferable that the thickness relationship is satisfied in the first region 31 and the second region 32. Consequently, it is possible to easily prevent excessive curving of the first thin portion U1 in the direction from the first region 31 toward the second region 32 and in the direction from the second region 32 toward the first region 31.

As illustrated in FIG. 6, the outer member 3 may include a body portion 33 enclosing a portion of the first tubular member 1 and a portion of the second tubular member 2; a first distal-end portion 34 located on the distal side of the body portion 33 and enclosing a distal end portion 1b of the first tubular member 1: and a second distal-end portion 35 located on the distal side of the body portion 33 and enclosing a distal end portion 2b of the second tubular member 2. Further, the second distal-end portion 35 may contain a material different from a material contained in the first distal-end portion 34 and from a material contained in the body portion 33. For example, when the second distal-end portion 35 includes a material that is more flexible than each of a material contained in the first distal-end portion 34 and a material contained in the body portion 33, damage to a biological tissue by a distal end portion of the catheter 91 can be easily avoided. Although a boundary line between the body portion 33 and the first distal-end portion 34 is illustrated in FIG. 6, the boundary line may disappear by welding.

As illustrated in FIG. 2, the first tubular member 1 may have the first opening portion 1E in communication with the lumen 1L. The first opening portion 1E can serve as an inlet or an outlet for the guidewire 9.

As illustrated in FIG. 2, the second tubular member 2 may have a second opening portion 2E in communication with the lumen 2L. The second opening portion 2E can serve as an inlet or an outlet for the guidewire 9.

As illustrated in FIG. 1, the handle portion 8 may be disposed at a proximal end portion la of the first tubular member 1. To be specific, it is preferable that the proximal end portion la of the first tubular member 1 is disposed in the handle portion 8. An operator can insert the catheter 91 into the body by holding and pushing the handle portion 8. The outer shape of the handle portion 8 may be any shape as long as an operator can hold the handle portion 8 with fingers. The handle portion 8 may be a resin molded product obtained by injection molding or the like. Examples of the resin include polyolefin-based resins such as polyethylene and polypropylene, polycarbonate-based resins, and (meth) acrylic-based resins. Among these resins, transparent resins such as polycarbonate and polymethyl methacrylate are preferable. Consequently, the inside of the handle portion 8 can be easily seen, and a wire can be easily inserted.

The proximal end 1A of the first tubular member 1 may be located in the handle portion 8. Consequently, the pushability can be improved.

The handle portion 8 may have a lumen extending in the longitudinal direction X to be in communication with the lumen 1L of the first tubular member 1 so that the guidewire 9 can be inserted through the lumen.

As illustrated in FIGS. 1 and 3, the catheter 91 may further include an outer tubular member 4 extending in the longitudinal direction X. The outer tubular member 4 may have a lumen 4L extending in the longitudinal direction X, and a portion of the first tubular member 1 may be disposed in the lumen 4L. A double-tube structure (coaxial structure) can be formed by the outer tubular member 4, and the transmissibility of a pushing force can be improved while the first tubular member 1 is protected. Specifically, it is preferable that the outer tubular member 4 includes, at a distal end portion, a fixed portion that is fixed to the first tubular member 1 and includes, on the proximal side of the fixed portion, a non-fixed portion that is not fixed to the first tubular member 1. In addition, the outer tubular member 4 may include, at a proximal end portion, a fixed portion fixed to the handle portion 8.

The outer tubular member 4 may be located on the proximal side of the proximal end 2A of the second tubular member 2. Consequently, the outer diameter of the outer tubular member 4 can be reduced.

The outer tubular member 4 may contain a resin that is the same as or harder than the resin of the outer member 3 or may be made of the harder resin. The harder resin can improve the pushability. In addition, the distal end of the outer tubular member 4 and the proximal end of the outer member 3 may be fixed to each other.

As illustrated in FIG. 2, on the proximal side of the proximal end of the first opening portion 1E, the first tubular member 1 may include a radiopaque marker 6A on the distal side of a point spaced apart by 1 cm toward the proximal side from the proximal end of the first opening portion 1E. Consequently, the position of the first opening portion 1E can be easily grasped. The radiopaque marker 6A may contain the same material as the radiopaque marker 6B or may be made of the same material as the radiopaque marker 6B. The shape of the radiopaque marker 6A may be different from that of the radiopaque marker 6B or may be the same as that of the radiopaque marker 6B.

Although not illustrated, the catheter 91 may further include a wire member extending in the longitudinal direction X and at least partially disposed in the outer tubular member 4. Consequently, transmissibility of a pushing force can be improved. The wire member may have a linear shape. The number of the wire members may be one or may be two or more. The proximal end of the wire member may be located in the handle portion 8. The distal end of the wire member may be located inside the outer tubular member 4 or the outer member 3. The wire member may be a solid member having no lumen. The wire member may contain a metal or may be made of a metal. The metal may be stainless steel, nickel titanium, cobalt chromium, or an alloy thereof. The wire member may include a fixed-shape portion extending in the longitudinal direction X and having a constant cross-sectional shape in the radial direction. It is more preferable that the wire member further includes a tapered portion located on the distal side of the fixed-shape portion and tapered toward the distal side.

The catheter 91 may be used for a treatment for expanding a stenotic or occluded site of a passage such as a blood vessel in the body or may be used by performing percutaneous coronary intervention (PCI) on, for example, a lesion in a bifurcated portion of a coronary artery.

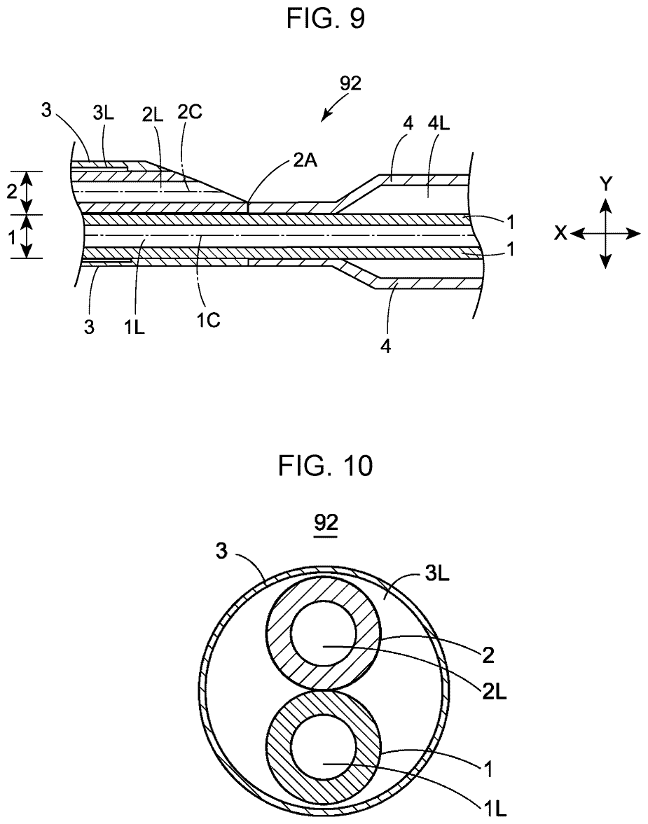

Hereinafter, a catheter according to a second embodiment will be described with reference to FIGS. 7 to 10. FIG. 7 is a side view of the catheter according to the second embodiment. FIG. 8 is a longitudinal sectional view of the region R1 in FIG. 7. FIG. 9 is a longitudinal sectional view of the region R2 in FIG. 7. FIG. 10 is a cross-sectional view of the catheter taken along line A-A in FIG. 7. Detailed description of portions common to the catheter according to the first embodiment will be omitted.

The catheter according to the second embodiment is a catheter 92 including the first tubular member 1 extending in the longitudinal direction X, the second tubular member 2 extending in the longitudinal direction X and adjacent to the first tubular member 1 in the radial direction Y, and the outer member 3 extending in the longitudinal direction X and including a portion that encloses at least a portion of the first tubular member 1 and at least a portion of the second tubular member 2. For details of each member, the description of the catheter 91 may be referred to.

As illustrated in FIGS. 7 to 10, in the catheter 92, the outer member 3 may have a tubular shape and have a lumen 3L extending in the longitudinal direction X, and at least a portion of the first tubular member 1 and at least a portion of the second tubular member 2 may be disposed in the lumen 3L. Consequently, the first tubular member 1 and the second tubular member 2 can be protected, and each tubular member can move to some extent due to a gap between the outer member 3 and the first tubular member 1 and a gap between the outer member 3 and the second tubular member 2. Therefore, the force of the guidewire 9 from the inside of the catheter 92 can be released, and as a result, puncture by the guidewire 9 can be easily prevented. A distal end portion of the tubular outer member 3 may be fixed to the first tubular member 1 and the second tubular member 2. A proximal end portion of the tubular outer member 3 may be fixed to the first tubular member 1 and the second tubular member 2. Consequently, the tubular outer member 3 is less likely to be wrinkled or twisted.

In the catheter 92, it is preferable that the first tubular member 1 and the second tubular member 2 are fixed to each other in the radial direction Y. Specifically, it is preferable that the outer surface of the first tubular member 1 and the outer surface of the second tubular member 2 are fixed to each other in the radial direction Y. the second tubular member 2 is less likely to be wrinkled or twisted. Examples of the fixing method include welding and adhesion.

The present application claims the benefit of priority based on Japanese Patent Application No. 2023-125555 filed on Aug. 1, 2023. The entire content of the description in Japanese Patent Application No. 2023-125555 filed on Aug. 1, 2023 is incorporated herein by reference.

Although the disclosure has been described with respect to only a limited number of embodiments, those skilled in the art, having benefit of this disclosure, will appreciate that various other embodiments may be devised without departing from the scope of the present disclosure. Accordingly, the scope of the invention should be limited only by the attached claims.

### REFERENCE SIGNS LIST

1 first tubular member
1a proximal end portion
1b distal end portion
1A proximal end
1B distal end
1C central axis
1E first opening portion
1L lumen
2 second tubular member
2b distal end portion
2A proximal end
2B distal end
2C central axis
2E second opening portion
2L lumen
3 outer member
3B distal end
3L lumen
4 outer tubular member
4L lumen
5 columnar body
6A, 6B radiopaque marker
8 handle portion
9 guidewire
31 first region
32 second region
33 body portion
34 first distal-end portion
35 second distal-end portion
91, 92 catheter
99 curved portion
T1 first thick portion
T1u thinned portion
T2 second thick portion
T2p raised portion
U1 first thin portion
U11 first portion
U12 second portion
U13 third portion
U1A proximal end
U1B distal end
X longitudinal direction
Y radial direction

1. A catheter comprising:
a first tubular member extending in a longitudinal direction;
a second tubular member extending in the longitudinal direction and adjacent to the first tubular member in a radial direction; and
an outer member extending in the longitudinal direction and including a portion that encloses at least a portion of the first tubular member and at least a portion of the second tubular member,
wherein a distal end of the first tubular member is located on a proximal side of a distal end of the second tubular member,
wherein a proximal end of the first tubular member is located on a proximal side of a proximal end of the second tubular member,
wherein a distal end of the outer member is located on a distal side of the distal end of the second tubular member, and
wherein the outer member includes:
a first thick portion,
a first thin portion located on a proximal side of the first thick portion and thinner than the first thick portion, and
a second thick portion located on a proximal side of the first thin portion and thicker than the first thin portion.

2. The catheter according to claim 1, wherein the first thick portion includes the distal end of the outer member.

3. The catheter according to claim 1, wherein the first thick portion and the first thin portion are adjacent to each other, and the first thin portion and the second thick portion are adjacent to each other.

4. The catheter according to claim 1, wherein the second thick portion includes a portion thicker than the first thick portion.

5. The catheter according to claim 1, wherein the second thick portion includes a raised portion raised in a direction away from the second tubular member.

6. The catheter according to claim 1, wherein a length of the first thin portion in the longitudinal direction is longer than a length of the first thick portion in the longitudinal direction.

7. The catheter according to claim 1, wherein a length of the first thin portion in the longitudinal direction is longer than a length of the second thick portion in the longitudinal direction.

8. The catheter according to claim 1, wherein the second tubular member includes a radiopaque marker located on a distal side of a proximal end of the first thin portion and on a proximal side of a distal end of the first thin portion.

9. The catheter according to claim 1, wherein the first thick portion includes a portion that becomes thinner toward the distal end of the outer member.

10. The catheter according to claim 1,
wherein the first thin portion includes:
a first portion,
a second portion located on a proximal side of the first portion and thinner than the first portion, and
a third portion located on a proximal side of the second portion and thicker than the second portion.

11. The catheter according to claim 1,
wherein the outer member includes a columnar body extending in the longitudinal direction £ and containing a resin, and
wherein at least a portion of the first tubular member and at least a portion of the second tubular member are disposed in the columnar body.

12. The catheter according to claim 1, wherein the outer member has a tubular shape and has a lumen extending in the longitudinal direction, and at least a portion of the first tubular member and at least a portion of the second tubular member are disposed in the lumen.

13. The catheter according to claim 1, wherein the outer member includes a body portion, a first distal-end portion, and a second distal-end portion, wherein:
the body portion encloses a portion of the first tubular member and a portion of the second tubular member,
the first distal-end portion is located on a distal side of the body portion and encloses a distal end portion of the first tubular member,
the second distal-end portion is located on a distal side of the body portion and encloses a distal end portion of the second tubular member, and
the second distal-end portion contains a material different from a material contained in the first distal-end portion and from a material contained in the body portion.

14. The catheter according to claim 1, wherein the first tubular member and the second tubular member are fixed to each other in the radial direction.

15. The catheter according to claim 1, wherein a handle portion is disposed at a proximal end portion of the first tubular member.
